(19) United States
(12) Patent Application Publication
(10) Pub. No.: US 2023/0193206 A1
(43) Pub. Date: Jun. 22, 2023

(54) Method for Producing Serum for Culturing Mammalian Cells

(71) Applicants: SAPPORO MEDICAL UNIVERSITY, Hokkaido (JP); NIPRO CORPORATION, Osaka (JP)

(72) Inventors: Osamu Honmou, Sapporo-shi, Hokkaido (JP); Yoshihiro Yoshikawa, Osaka (JP); Takanori Sambuichi, Osaka (JP); Naomi Morikawa, Osaka (JP); Kazunari Abe, Osaka (JP)

(73) Assignees: SAPPORO MEDICAL UNIVERSITY, Hokkaido (JP); NIPRO CORPORATION, Osaka (JP)

(21) Appl. No.: 17/789,314

(22) PCT Filed: Dec. 25, 2020
(86) PCT No.: PCT/JP2020/048984

§ 371 Date: Jun. 27, 2022

(30) Foreign Application Priority Data
Dec. 27, 2019 (JP) 2019-237738

(51) Int. Cl. C12N 5/0775

(52) U.S. Cl. CPC C12N 5/0663; C12N 2509/00; C12N 2500/84

(57) ABSTRACT

Provided is a method for producing serum that exhibits improved cell proliferation activity. A method for producing serum for culturing mammalian cells, comprising: maintaining blood collected from a first mammal at a temperature between 15° C. and 25° C. for up to 48 hours and preparing serum from the blood.

### TECHNICAL FIELD

The invention relates to a method for producing serum for culturing mammalian cells. The invention also relates to a method for producing a medicament including cultured mammalian cells.

### BACKGROUND

Regenerative medicine for the central nervous system has been recently investigated and developed. Regenerative medicine in which stem cells are used includes cell therapy for replacing damaged cells. Cell therapy for treating cranial nerve disease includes harvesting cells from a tissue, the cells being capable of differentiating into nervous system cells, culturing the cells, and then transplanting the cultured cells to an individual (Patent Literature 1).

Cell therapy for acute phase injury such as spinal cord injury due to accidents essentially needs a rapid and mass production of cultured cells, the cells (donner cells) being given by a donner. In vitro cultivation of cells in a culture medium may use a growth-promoting factor such as serum to improve cell survival and increase the proliferation rate of donor cells.

Sera are often used to grow desired cells efficiently in the field of cell therapy. The serum used in producing a cell therapy medicament is preferably autologous serum of a subject to whom the medicament including the cultured cells is administered and enters the body together because the autologous serum has little effect on the subject. However, because human serum alone does not have the desired cell growth-promoting activity, fetal bovine serum (FBS) or other growth factors are added (Patent Literatures 2 and 3).

Medical facilities that collect blood, the source of autologous serum, from subjects rarely have the equipment to prepare serum in sufficient quantity and quality to produce cell therapeutics. The cell therapeutics are usually manufactured there after the blood is transported to a medical cell culture manufacturing facility that meets prescribed standards (for example, Cell Processing Center (CPC) that meets GMP standards). This requires transporting blood from the medical facility to the manufacturing facility. Depending on the facility's location, long-term transportation may be required, and blood quality may deteriorate. A method for adding a preservative to the blood is proposed to prevent deterioration of blood quality from long-term transportation (Patent Literature 4).

### CITATION LIST

### Patent Literature 1: WO02/00849 A1

### Patent Literature 2: JP H10-179148 A

### Patent Literature 3: JP 2003-235548 A

### Patent Literature 4: 2015-151334 A

### SUMMARY

Adding growth factors derived from other species to medicaments including human serum may result in the desired cell growth-promoting activity. However, the addition of growth factors derived from other species is laborious and costly for safety testing. Therefore, there is a demand in the field for a method for producing serum that exhibits the desired cell proliferation activity without adding growth factors derived from other species.

In cell therapy using autologous cells grown in a medium containing autologous serum, there is a limit to the amount of blood that can be collected at one time from the viewpoint of maintaining the health of the subject. Therefore, there is a demand in the field for a method for producing serum exhibiting the desired cell proliferation activity from the amount of blood collected.

Adding a preservative provides shelf life for long-term transport. The preservative added to a cell therapeutic also enters the subject's body together with the therapeutic. Therefore, from a safety viewpoint, there is a demand for a method for producing serum that exhibits the desired cell proliferation activity without using a preservative.

### Solution to Problem

The inventors found that by maintaining blood collected from the individual at a temperature within a predetermined range and keeping the temperature within a predetermined time, the serum prepared from the blood exhibited the desired cell proliferative activity, and made the invention.

A first aspect of the invention provides a method for producing serum for culturing mammalian cells, the method comprising: maintaining blood collected from a first mammal at a temperature between 15° C. and 25° C. for up to 48 hours and preparing serum from the blood. A second aspect of the invention provides a method for producing a medicament comprising cultured mammalian cells, the method comprising culturing mammalian cells in a culture medium, wherein the culture medium contains serum for culturing the mammalian cells; and the serum is prepared from blood collected from a first mammal by maintaining the blood at a temperature between 15° C. and 25° C. for up to 48 hours.

### Effects

The invention provides a method for producing serum that exhibits improved cell proliferation activity and a method for producing a medicament, comprising culturing mammalian cells with the serum produced by the above method.

### BRIEF DESCRIPTION OF DRAWINGS

FIG. 1 is a bar graph showing the proliferation rates of cells cultured with sera prepared from blood kept at 10° C., 20° C., or 25° C. for a predetermined time.

FIG. 2 is a bar graph showing the proliferation rates of cells cultured with sera prepared from blood kept at 15° C., 20° C., or 25° C. for a predetermined time.

### DESCRIPTION OF EMBODIMENTS

“Blood” in the specification refers to a body fluid that circulates the body of a mammal and includes blood cell components such as red blood cells, white blood cells, platelets, and the like. Blood collected from a mammal may be, for example, venous blood, arterial blood, or peripheral blood. In an embodiment, blood collected from a mammal is peripheral blood. Blood can be, for example, collected from a mammal according to known methods. For example, blood can be collected using a syringe needle and a blood collection container. In an embodiment, blood for preparing serum is free of preservatives.

A “blood collection container” in the specification refers to any container capable of aseptically holding blood collected from a mammal. A blood collection container may be, for example, any container known in the field related to collecting blood and commercially available. The blood collection container may be, for example, any bag or tube for collecting blood. For example, the blood collection container may be the device for holding blood described in US2013/0130382, the content of which is incorporated herein by reference.

“Serum” in the specification refers to a liquid part separated from blood clots formed by coagulation of mammalian blood. Serum can be, for example, produced from blood collected from a mammal according to known methods. Serum can be, for example, used by adding it to a medium for culturing mammalian cells with a dispensing tool.

“Blood clots” in the specification refer to biological structures formed during the coagulation process of mammalian blood. Blood clots include, but are not limited to, blood cell components and fibrin. For example, according to known methods, blood clots can be formed by placing mammalian blood aseptically with no anticoagulant in a container at room temperature (e.g., 22° C. to 27° C.).

“Serum for culturing mammalian cells” in this specification refers to serum obtained by keeping blood collected from a mammal at a temperature between 15° C. and 25° C. for up to 48 hours. Keeping the blood at a temperature between 15° C. and 25° C. may be, for example, to place a container holding the blood at a specific temperature in the range from 15° C. to 25° C. Keeping the blood at a temperature between 15° C. and 25° C. may be, for example, to place the blood inside a temperature-controllable container, device, or combination thereof capable of adjusting the temperature at a specific temperature or a specific temperature range from 15° C. to 25° C. Blood kept at a temperature between 15° C. and 25° C. may be, for example, static or transported.

A “temperature-controllable container or device” in the specification refers to a container or device with a function to adjust the temperature inside the container or device to a predetermined temperature range. The temperature-controllable container or device may be, for example, a container or device that is covered with thermal insulation material and is not susceptible to the effects of ambient temperature. The thermal insulation material may be, for example, styrofoam, rigid polyurethane foam, vacuum insulation material (e.g., a structure that contains a glass wool core or carbon material inside a gas barrier film and the inside of the film package is evacuated and sealed), or a combination thereof. The temperature-controllable container may include inside the container, for example, heat storage medium, exothermic agent, material for cold storage, cold pack, or temperature control unit (e.g., a small cooling device with Peltier element controlled by thermostat) or a combination thereof. The temperature-controllable devices may be, for example, equipped with a temperature control unit (e.g., a small cooling device equipped with Peltier element controlled by thermostat) and optionally heat storage medium or material for cold storage. The temperature-controllable device may be, for example, transportation means (e.g., car, boat, or airplane) with a function to adjust the temperature. The combination of temperature-controllable container and device may be, for example, a temperature-controllable device in which a temperature-controllable container containing a container that holds the blood is contained.

Blood collected from a mammal can be placed inside a temperature-controllable container, device, or combination thereof such that the temperature is adjusted, for example, in a range from 15° C. to 24° C., from 15° C. to 23° C., from 15° C. to 22° C., from 15° C. to 21° C., from 15° C. to 20° C., from 16° C. to 25° C., from 16° C. to 24° C., from 16° C. to 23° C., from 16° C. to 22° C., from 16° C. to 21° C., from 16° C. to 20° C., from 17° C. to 25° C., from 17° C. to 24° C., from 17° C. to 23° C., from 17° C. to 22° C., from 17° C. to 21° C., from 17° C. to 20° C., from 18° C. to 25° C., from 18° C. to 24° C., from 18° C. to 23° C., from 18° C. to 22° C., from 18° C. to 21° C., from 18° C. to 20° C., from 19° C. to 25° C., from 19° C. to 24° C., from 19° C. to 23° C., from 19° C. to 22° C., from 19° C. to 21° C., or from 19° C. to 20° C.

In an embodiment, blood collected from a mammal is placed inside a temperature-controllable container, device, or combination thereof such that the temperature is adjusted, for example, at a specific temperature of 15° C., 16° C., 17° C., 18° C., 19° C., 20° C., 21° C., 22° C., 23° C., 24° C., or 25° C. A specific temperature refers to a temperature range, including a temperature control error of the temperature-controllable container. When a temperature control error of the temperature-controllable container, device, or combination thereof is, for example, ±0.5° C., the specific temperature being 20° C. is 19.5° C. to 20.5° C.

In an embodiment, blood collected from a mammal is placed inside a temperature-controllable container, device, or combination thereof such that the temperature is adjusted in a range from 17° C. to 23° C., from 18° C. to 22° C., or from 19° C. to 21° C.

“Keeping time” or “time kept” at a specific temperature in the specification refers to a period from the time blood collected from a mammal is placed inside a temperature-controllable container, device, or combination thereof to the time the blood is taken out from there. The keeping time is not limited as long as it is up to 48 hours, for example, no longer than 45 hours, no longer than 40 hours, no longer than 35 hours, no longer than 30 hours, or no longer than 24 hours. The temperature kept at a specific temperature for the time may be checked, for example, by placing a temperature recording device together with blood collected from a mammal.

In an embodiment, the time from collecting blood from a mammal to placing the blood in a temperature-controllable container, device, or combination thereof is no longer than 1 hour, no longer than 0.5 hours, no longer than 0.3 hours, no longer than 15 minutes, no longer than 10 minutes, no longer than 5 minutes, or no longer than 3 minutes.

In an embodiment, the step of preparing serum comprises steps of: placing the mammalian blood taken out from the temperature-controllable container, device, or combination thereof; separating the blood into blood clots and a liquid part; and collecting the liquid part. The step of placing may comprise, for example, keeping the blood at room temperature (e.g., 21° C. to 27° C.). The keeping time in the step of placing may be, for example, until the formation of blood clots can be found or within the predetermined time (any period from 0.5 hours to 2 hours). For example, the step of placing may comprise further placing the blood when the formation of blood clots cannot be found in the blood kept at room temperature for the predetermined period (e.g., 2 hours) after the blood is taken out from the temperature-controllable container, device, or combination thereof. The formation of blood clots may be, for example, visually checked.

In the step of collecting, serum can be, for example, collected from blood clots according to known methods. The step of collecting serum may comprise, for example, transferring a liquid part separated from the blood clots to another container with an instrument such as a pipette; transferring the supernatant after precipitating the blood clots by centrifugation to another container with an instrument such as a pipette; or filtering a mixture of the blood clots separated into the liquid part through a filter and collecting the filtrate as serum.

In an embodiment, the method for producing serum further comprises examining an aliquot of the blood collected from a mammal or an aliquot of prepared serum for either one or both of a tumor marker and an infectious factor. In an embodiment, the method for producing serum comprises examining an aliquot of the blood collected from a mammal for either one or both of a tumor marker and an infectious factor. In these examinations, each marker or factor can be measured according to known determination methods, and the measurements may be compared to a standard or reference value regarding the respective marker or factor so that the aliquot can be determined to be positive or negative.

The serum tumor marker includes, for example, one, two, or more selected from the group consisting of ferritin, CEA, AFP, BFP, CA125, CA15-3, CA19-9, CA72-4, STN, DUPAN-2, SLX, ST-439, SPAN-1, SCC, PSA, G-seminoprotein, TPA, CYFRA, PAP, NSE, C-peptide, PIVKA, Pro-GRP, HCGB, elastase, β2-microglobulin, S-NTX, anti-p53 antibody, and HER2. The infectious factor includes, for example, one, two, or more selected from the group consisting of HIV, ATL, HBV, HCV, Treponema pallidum, and human parvovirus B19.

In an embodiment, the method for producing serum may be continued when in the serum production method, all serum tumor markers examined are negative and all infection factors examined are negative. In other embodiments, the method for producing serum may be discontinued when in the serum production method, any one of the serum tumor markers examined is positive or any one of the infectious factors examined is positive.

A “mammal” in the specification refers to any mammalian individual. The mammal may include, but not be limited to, human or non-human mammals. The non-human mammal may include, but not be limited to, a rodent such as a mouse, rat, guinea pig, and hamster; non-human primate such as a chimpanzee; Artiodactyla such as a cow, goat, and sheep; Perissodactyla such as a horse; a pet animal such as a rabbit, dog, and cat. In an embodiment, the mammal is a horse, pet animal, non-human primate, or human. In an embodiment, the mammal is human.

A “first mammal” in the context of collecting blood to produce serum, a “second mammal” in the context of mammalian cells, and a “third mammal” in the context of pharmaceutical administration may be the same individual or different individuals. If the first mammal, the second mammal, and the third mammal are different individuals, the first mammal, the second mammal, and the third mammal may be the same species (homozoic) or different species (heterozoic). In a preferred embodiment, the first mammal, the second mammal, and the third mammal are the same species. In a more preferred embodiment, the first mammal, the second mammal, and the third mammal are the same individual.

“Stem cells” in the specification refers to any cells with both self-renewal and differentiation capabilities. “Self-renewal capability” in the specification refers to the capability to continuously produce stem cells having the same capability as self as at least one daughter cell through cell division. “Differentiation capability” in the specification refers to the capability to divide into specialized cells different from the self.

The stem cell may include, but are not limited to, pluripotent stem cell capable of differentiating into multiple types of cells belonging to each of the three germ layers, endoderm, ectoderm, and mesoderm; multipotent stem cell capable of differentiating into multiple types of cells, which are restricted to one germ layer; and unipotent stem cell capable of differentiating into only one cell type. Pluripotent stem cells may be, for example, embryonic stem cells or recombinant cells (induced pluripotent stem cells) genetically engineered from somatic cells to give them pluripotency. Multipotent stem cells may be, for example, hematopoietic or mesenchymal stem cells. Unipotent stem cells may be, for example, germline stem cells. Stem cells can be obtained from biological samples of mammalian origin according to known methods or may be commercially available.

“Mesenchymal stem cells” in the specification are non-hematopoietic adherent cells capable of self-renewal and differentiation into connective tissue cells such as osteocytes, chondrocytes, and adipocytes as well as into neurons, cardiomyocytes, and liver cells. Mesenchymal stem cells are known to be present in bone marrow fluid in very small amounts (0.001-0.01% of the population of cells having a nucleus).

“Biological sample” in the specification refers to a portion of tissue or body fluid of a mammal. The biological sample may be, for example, a portion of tissue or body fluid itself obtained from the mammal, or a sample that has undergone appropriate treatment (e.g., removal of unnecessary components, purification of specific cell fractions, enzymatic treatment, and preservation treatment) as needed. The body fluid may be, for example, bone marrow fluid, blood (peripheral or cord blood), or lymphatic fluid. The tissue may be, for example, muscle tissue, bone tissue, skin, lymphoid tissue, vascular or digestive tissue, or an embryo (e.g., mammalian embryos except for human embryos).

The biological sample can be obtained from the mammal according to known methods. The collection of the biological sample can be obtained, for example, by anesthetizing (local or general anesthesia) a second mammal from which the bone marrow fluid is obtained, inserting a needle into the sternum or iliac bone, and aspirating the bone marrow fluid as the biological sample with a syringe. The collection of the biological sample can be obtained, for example, by inserting a needle directly into the umbilical cord and aspirating the umbilical cord blood as the biological sample with a syringe.

“Mammalian cells” in the specification refers to any cells from a mammal that can proliferate by cell culture. The mammalian cells may be, for example, cells present in a biological sample collected from the mammal or recombinant cells originating from the mammalian and generated by genetic engineering. The mammalian cells may be, for example, cells that can be attached to a culture vessel and cultured or cells that can be cultured in a suspended condition. For example, mammalian cells may be cells present in a biological sample collected from the mammal, e.g., somatic cells or stem cells. Somatic cells may be, for example, one, two, or more of osteoblasts, fibroblasts, epithelial cells, and vascular endothelial cells. Stem cells include, for example, at least one type of mesenchymal stem cell.

In an embodiment, the mammalian cells are stem cells. In an embodiment, the mammalian cells are mesenchymal stem cells. In a preferred embodiment, the mammalian cells are human mesenchymal stem cells. In a more preferred embodiment, the mammalian cells are bone marrow-derived human mesenchymal stem cells. Bone marrow-derived mesenchymal stem cells can be obtained, for example, by proliferating in cell culture mesenchymal stem cells present in bone marrow fluid collected from humans.

“Medicament” comprising cultured mammalian cells in the specification refers to any composition for treating a disease or disorder that may respond to cell therapy. Medicaments are required to meet various criteria for medical products, such as those related to impurities derived from the manufacturing process. The medicament may include, but is not limited to, a pharmaceutically acceptable diluent, excipient and/or base material, depending on the application. The medicament may be, for example, injectable (liquid) and implantable (e.g., sheet) forms. The medicament is appropriately delivered to a site to be treated depending on the form of the medicament. The method for delivery of the medicament may be, for example, local implantation by surgical means, intravenous administration, lumbar puncture administration, local injection, subcutaneous administration, intradermal administration, intraperitoneal administration, intramuscular administration, intracerebral administration, intraventricular administration, or intravenous administration. In an embodiment, the medicament is in injectable form and is administered intravenously. The medicament in the injectable form may be, for example, for bolus or drip administration.

The medicament can be used, for example, in tissue repair or tissue regeneration. The medicament is used to treat neurological diseases. Neurological disorders include, but are not limited to, central and peripheral demyelinating disease; central and peripheral degenerative disease; stroke (including cerebral infarction, cerebral hemorrhage, and subarachnoid hemorrhage); brain tumor; higher brain dysfunction including dementia; mental disorders; epilepsy; traumatic neurological disorder (including head injury, brain contusion, and spinal cord injury); and spinal code infarction.

The medicament is not limited to treating neurological diseases and can be used, for example, to treat a disease associated with abnormalities in myocardium, joint, cartilage, liver, bone marrow, and other tissues. The medicament can be used to treat, for example, spinal cord injury, stroke, dementia, or spinal cord infarction. The medicament may be useful for the above diseases, which is supported by, for example, US2010/254953, the content of which is incorporated herein by reference.

Cultured mammalian cells used for the medicament can be produced by culturing mammalian cells in a medium containing serum prepared by the method for producing serum described herein, according to known cell-culturing methods. Such a production method is described, for example, in JP2002-100662 A, the content of which is incorporated herein by reference.

“Culture medium” in the specification refers to any composition allowing mammalian cells in vitro to keep survival and divide cells. The culture medium includes serum prepared by the method for producing serum described herein. The culture medium can be produced using a base medium for culturing cells, which is mainly composed of amino acids, vitamins, and electrolytes. The base medium may be, for example, Dulbecco's modified Eagle's medium (DMEM), NPBM, and αMEM. In an embodiment, the culture medium is DMEM. The culture medium can be prepared according to known methods and is commercially available.

The culture medium contains, in a base medium, serum prepared by the method for producing serum described herein at 1 to 20% volume, preferably 5 to 15% volume, more preferably 8 to 12% volume per volume of the medium. The culture medium may be used, for example, with antibiotics commonly used in the field of cell culture (e.g., penicillin, streptomycin, and the like), either alone or in combination. For example, the culture medium may contain each penicillin and streptomycin at 0.5 to 2% volume, preferably 0.8 to 1.2% volume per volume of the medium. In an embodiment, the culture medium may contain an anticoagulant at no more than 0.02 U/mL per volume of the medium.

Growth factors and/or differentiation factors may be added to the base medium as needed. The growth and differentiation factors are selected according to the desired differentiation process and state, the required growth rate, and the like. For example, the factors may include vitamins such as ascorbic acid and nicotinamide; neurotrophic factors such as NGF and BDNF; osteogenic factors such as BMP; epidermal cell growth factors; basic fibroblast growth factors; insulin-like growth factors; cytokines such as IL-2. In an embodiment, the culture medium does not contain serum prepared from blood of a different species (heterozoic) other than the serum prepared by the method for producing serum described herein.

Mammalian cells can be cultured under conditions known in the field of cell culture. Mammalian cells can be cultured, for example, under 37° C. and 5% CO₂. Mammalian cells can be cultured with any culture vessels of any volume and shape. The volume or form of the cultured mammalian cells can be controlled by the size or shape of the culture vessel's surface to which cells adhere. Culturing mesenchymal stem cells is described, for example, in JP2012-100662 A, the content of which is incorporated herein by reference.

“Cells do not substantially contact with anticoagulant” in the specification means that the amount of anticoagulant used is substantially reduced throughout the entire period from collecting mammalian cells to culturing the mammalian cells. For example, the anticoagulant is added in an amount that the inner surface of a container (such as a blood collection tube) for collecting mammalian cells wets with an anticoagulant solution; is not added; or is added in an amount that remains when the anticoagulant in the sample is substantially removed at the start of culture. The amount of anticoagulant added is small when collecting a biological sample to proliferate mammalian cells more rapidly and in large quantities.

In an embodiment, the amount of anticoagulant added to a biological sample collected from the second mammal (i.e., pre-added to a container holding the collected biological sample) is less than 0.2 U/mL per volume of the biological sample.

“Anticoagulant” in the specification refers to a substance that inhibits the adhesion of cells to the extracellular matrix, cell to cell, or cells to a substrate. The adhesion is inhibited by the anticoagulant in a biological sample binding to the cell surface and interacting with proteins exerting a blood coagulation effect, the proteins being in the extracellular matrix. The anticoagulant includes, for example, heparin and heparin derivatives (e.g., glycosaminoglycan, which is desulfated at the 6-position of D-glucosamine being a part of heparin).

Embodiments of the invention are, for example, described below:

[Item 1] A method for producing serum for culturing mammalian cells, the method comprising: maintaining blood collected from a first mammal at a temperature between 15° C. and 25° C. for up to 48 hours and preparing serum from the blood.

[Item 2] The method according to claim 1, wherein the first mammal is a human.

[Item 3] The method according to item 1 or 2, further comprising examining an aliquot of the blood or prepared serum for either one or both of a tumor marker and an infectious factor.

[Item 4] The method according to any one of items 1 to 3, wherein the mammalian cells are cells derived from a second mammal of the same species as the first mammal.

[Item 5] The method according to any one of items 1 to 4, wherein the mammalian cells are cells derived from the same mammal as the first mammal.

[Item 6] The method according to any one of items 1 to 5, wherein the mammalian cells are stem cells.

[Item 7] The method according to any one of items 1 to 6, wherein the mammalian cells are bone marrow-derived mesenchymal stem cells.

[Item 8] A method for producing a medicament comprising cultured mammalian cells, the method comprising culturing mammalian cells in a culture medium, wherein
the culture medium contains serum for culturing the mammalian cells, and
the serum is prepared from blood collected from a first mammal by maintaining the blood at a temperature between 15° C. and 25° C. for up to 48 hours.

[Item 9] The method according to item 8, wherein the first mammal is a human.

[Item 10] The method according to item 8 or 9, wherein the serum is examined for either one or both of a tumor marker and an infectious factor.

[Item 11] The method according to any one of items 8 to 10, wherein the culture medium contains the serum at 1 to 20% per medium volume.

[Item 12] The method according to any one of items 8 to 11, wherein the culture medium contains anticoagulant at less than 0.02 U/mL per medium volume.

[Item 13] The method according to any one of items 8 to 12, wherein the mammalian cells are cells derived from a second mammal of the same species as the first mammal.

[Item 14] The method according to any one of items 8 to 13, wherein the mammalian cells are cells derived from the same mammal as the first mammal.

[Item 15] The method according to any one of items 8 to 14, wherein the mammalian cells are stem cells.

[Item 16] The method according to any one of items 8 to 15, wherein the mammalian cells are bone marrow-derived mesenchymal stem cells.

[Item 17] The method according to any one of items 8 to 16, wherein the mammalian cells are cells derived from the same mammal as a third mammal in need of administration of the medicament.

[Item 18] The method according to any one of items 8 to 17, wherein the mammalian cells are collected in a condition where anticoagulant is added to a biological sample collected from the second mammal at less than 0.2 U/ml per sample volume so that the mammalian cells do not substantially contact with the anticoagulant.

[Item 19] The method according to any one of items 12 to 18, wherein the anticoagulant is heparin, heparin derivative, or salt thereof.

[Item 20] The method according to any one of items 8 to 19, wherein the medicament is for treating spinal cord injury, stroke, dementia, or spinal cord infarction.

[Item 21] The method according to any one of items 8 to 20, further comprising collecting the mammalian cells cultured in the culture medium.

Specific examples are described below. However, these examples are merely preferred embodiments of the invention and do not limit the scope of the invention recited in the accompanying claims.

### EXAMPLE

[Preparation of Serum]

(Control Sample)

Peripheral blood was collected from volunteers in plastic centrifuge tubes. The collected blood was allowed to place at room temperature to separate the blood clots from the liquid part. The blood clots were precipitated by centrifugation at 2380×g at room temperature, and the supernatant was placed in a sterile plastic container to prepare serum.

### Comparative Examples 1-3

Serum was prepared in the same manner as the control sample, except that the collected blood was allowed to place at 10° C. for 9 hours (Comparison Example 1), 24 hours (Comparison Example 2), or 48 hours (Comparison Example 3).

### Examples 1-3

Serum was prepared in the same manner as the control sample, except that the collected blood was allowed to place at 20° C. for 9 hours (Example 1), 24 hours (Example 2), or 48 hours (Example 3).

### Examples 4-6

Serum was prepared in the same manner as the control sample, except that the collected blood was allowed to place at 25° C. for 9 hours (Example 4), 24 hours (Example 5), or 48 hours (Example 6).

The time required to separate the blood clots from the liquid part of serum prepared from the blood in Comparative Examples 1-3 tended to be longer than the time required to separate the liquid part of serum prepared from the blood of Examples 1-6.

[Preparation of Culture Media]

DMEM (88 v/v %), L-glutamine solution (1 v/v %), penicillin-streptomycin (1 v/v %), and each serum (10 v/v %) were mixed and then sterilized by filtrating the mixture through a filter (0.2 μm) to prepare Culture Medium A.

[Culturing Mammalian Cells]

Mesenchymal stem cells (LONZA KK) were added to Culture Medium A, previously kept at 37° C., and were seeded at 2.0*10⁵ cells per 100 mm culture dish. The cells were cultured at 37° C. under 5% CO₂ condition to allow the cells to adhere to the culture dish substrates, and then the culture medium was aspirated to remove suspended components. The culture dishes were washed using phosphate-buffered saline. After washing, Culture Medium A was added, and cells adhered to the culture dish substrates were further cultured. After cells reached about 80% confluence, cells were subcultured. Subculturing was carried out by collecting cultured cells from the dishes with a stripping solution containing trypsin and EDTA (ethylenediaminetetraacetic acid), suspending the collected cells in Culture Medium A, and seeding 2.0*10⁵ cells per 100 mm culture dish.

Cells seeded to culture dishes were cultured at 37° C. under 5% CO₂ condition in the same manner as the above. After cells reached about 80% confluence, the cultured cells (2nd subculturing) were collected and counted.

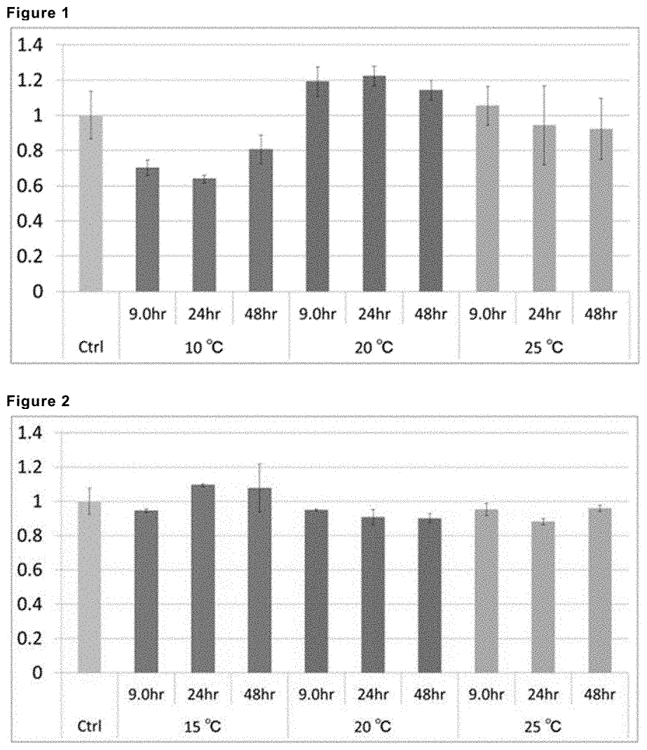

The number of cells cultured with each serum is shown in Table 1. Their relative values compared to the number of cells cultured with the control serum can be seen in FIG. 1. The relative values in FIG. 1 correspond to the proliferation rate of the cells of the 2nd subculturing to the cells of the control.

TABLE 1

| | Temperature during | Time during | | 2nd |
|---|---|---|---|---|
| | transportation | transportation | | subculturing |
| Control | | — | | 5.27*10⁵ |
| Comparative | 10° C. | 9 | hr | 3.70*10⁵ |
| Example 1 | | | | |
| Comparative | | 24 | hr | 3.37*10⁵ |
| Example 2 | | | | |
| Comparative | | 48 | hr | 4.24*10⁵ |
| Example 3 | | | | |
| Example 1 | 20° C. | 9 | hr | 6.28*10⁵ |
| Example 2 | | 24 | hr | 6.44*10⁵ |
| Example 3 | | 48 | hr | 6.02*10⁵ |
| Example 4 | 25° C. | 9 | hr | 5.55*10⁵ |
| Example 5 | | 24 | hr | 4.97*10⁵ |
| Example 6 | | 48 | hr | 4.86*10⁵ |

Collected blood was used to prepare serum by placing the blood at 15° C. for the predetermined hours (Examples 7 to 9), serum by placing the blood at 20° C. for the predetermined hours (Examples 10 to 12), serum by placing the blood at 25° C. for the predetermined hours (Examples 13 to 15), and serum without placing the blood (control) according to the same manner at the above. Culture Medium A including each prepared serum was used to culture Mesenchymal stem cells (LONZA KK) and subculture them (2nd subculturing). The proliferation rates of the 2nd subcultured cells in Examples 10 to 15, which were compared to cells cultured with the control serum, can be seen in FIG. 2.

TABLE 2

| | Temperature during | Time during | | 2nd |
|---|---|---|---|---|
| | transportation | transportation | | subculturing |
| Control | | — | | 4.25*10⁵ |
| Example7 | 15° C. | 9 | hr | 4.02*10⁵ |
| Example8 | | 24 | hr | 4.66*10⁵ |
| Example9 | | 48 | hr | 4.58*10⁵ |
| Example10 | 20° C. | 9 | hr | 4.04*10⁵ |
| Example11 | | 24 | hr | 3.86*10⁵ |
| Example12 | | 48 | hr | 3.83*10⁵ |
| Example13 | 25° C. | 9 | hr | 4.05*10⁵ |
| Example14 | | 24 | hr | 3.75*10⁵ |
| Example15 | | 48 | hr | 4.08*10⁵ |

FIG. 1 shows that the cell proliferation rates obtained with the use of sera, prepared from the blood that was kept at 10° C. (Comparative Examples 1-3), were lower than the proliferation rate obtained with the control serum (not transported). FIGS. 1 and 2 show that in the cell culturing with sera prepared from the blood that was kept at 20° C. or 25° C., the cell proliferation rates remained almost unchanged regardless of the long or short time kept at the temperature (FIG. 2) or tended to decrease as the time kept at the temperature increased (FIG. 1). FIG. 2 shows that in the cell culturing with sera prepared from the blood that was kept at 15° C., the cell proliferation rates tended to increase as the time kept at the temperature increased. Based on the results shown in FIGS. 1 and 2, the inventors found that the serum, prepared from the blood that was kept at least at a temperature between 15° C. and 25° C. for up to 48 hours, can prevent the decrease in cell proliferation rate as observed with the use of the serum prepared from the blood kept at a relatively low temperature (10° C.).

1. A method for producing serum for culturing mammalian cells, the method comprising: maintaining blood collected from a first mammal at a temperature between 15° C. and 25° C. for up to 48 hours and preparing serum from the blood.

2. The method according to claim 1, wherein the first mammal is a human.

3. The method according to claim 1, further comprising examining an aliquot of the blood or prepared serum for either one or both of a tumor marker and an infectious factor.

4. The method according to claim 1, wherein the mammalian cells are cells derived from a second mammal of the same species as the first mammal.

5. The method according to claim 1, wherein the mammalian cells are cells derived from the same mammal as the first mammal.

6. The method according to claim 1, wherein the mammalian cells are stem cells.

7. The method according to claim 1, wherein the mammalian cells are bone marrow-derived mesenchymal stem cells.

8. A method for producing a medicament comprising cultured mammalian cells, the method comprising culturing mammalian cells in a culture medium, wherein
the culture medium contains serum for culturing the mammalian cells, and
the serum is prepared from blood collected from a first mammal by maintaining the blood at a temperature between 15° C. and 25° C. for up to 48 hours.

9. The method according to claim 8, wherein the first mammal is a human.

10. The method according to claim 8, wherein the serum is examined for either one or both of a tumor marker and an infectious factor.

11. The method according to claim 8, wherein the culture medium contains the serum at 1 to 20% per medium volume.

12. The method according to claim 8, wherein the culture medium contains anticoagulant at less than 0.02 U/mL per volume of the medium.

13. The method according to claim 8, wherein the mammalian cells are cells derived from a second mammal of the same species as the first mammal.

14. The method according to claim 8, wherein the mammalian cells are cells derived from the same mammal as the first mammal.

15. The method according to claim 8, wherein the mammalian cells are stem cells.

16. The method according to claim 8, wherein the mammalian cells are bone marrow-derived mesenchymal stem cells.

17. The method according to claim 8, wherein the mammalian cells are cells derived from the same mammal as a third mammal in need of administration of the medicament.

18. The method according to claim 8, wherein the mammalian cells are collected in a condition where anticoagulant is added to a biological sample collected from the second mammal at less than 0.2 U/ml per volume of the sample so that the mammalian cells do not substantially contact with the anticoagulant.

19. The method according to claim 8, wherein the anticoagulant is heparin, heparin derivative, or salt thereof.

20. The method according to claim 8, wherein the medicament is for treating spinal cord injury, stroke, dementia, or spinal cord infarction.
